(19) United States
(12) Patent Application Publication
(10) Pub. No.: US 2020/0129395 A1
(43) Pub. Date: Apr. 30, 2020

(54) STYLING PRODUCT FOR HEAD AND/OR BEARD HAIR I

(71) Applicant: Henkel AG & Co. KGaA, Duesseldorf (DE)

(72) Inventors: MARCUS NOLL, Quickborn (DE); SANDRA BRANDT, Pinneberg (DE); NORA KOOPMANN, Hamburg (DE)

(73) Assignee: Henkel AG & Co. KGaA, Duesseldorf (DE)

(21) Appl. No.: 16/654,966

(22) Filed: Oct. 16, 2019

(30) Foreign Application Priority Data
Oct. 29, 2018 (DE) 10 2018 218 513.1

(51) Int. Cl. A61K 8/34; A61K 8/37; A61K 8/04; A61K 8/92; A61Q 5/06

(52) U.S. Cl. CPC A61K 8/342; A61K 8/375; A61Q 5/06; A61K 8/04; A61K 8/92; A61K 8/345

(57) ABSTRACT

The application describes a cosmetic composition for temporary deformation of hair, comprising, in each case based on the total weight of the composition, a) from about 20 to about 60% by weight of water, b) from about 0.5 to about 20% by weight of at least one oil, and c) from about 20 to about 65% by weight of at least one alkoxylated fatty alcohol.

### CROSS-REFERENCE TO RELATED APPLICATION

This application claims priority to German Patent Application No. 10 2018 218 513.1, filed Oct. 29, 2018, which is incorporated herein by reference in its entirety.

### TECHNICAL FIELD

The present application describes a cosmetic composition for temporary deformation of hair, which comprises water, oil and an alkoxylated fatty alcohol and their use. In addition, a method for temporary deformation of hair using the cosmetic composition is disclosed.

### BACKGROUND

For some time, the pomade has been celebrating a renaissance in men's care with the comeback of the full beard and barbershops.

Pomade has been around since the 18th century and was obtained from apples in a complex process (Italian: pomo or French: pomme).

Pomades are primarily developed to give the hair a smooth look while providing a shiny, but not oily, finish. In principle, pomades are particularly suitable for hairstyles that are shaped with a comb. Unlike gels, pomades do not dry out or do not leave hard film on the hair. The hair can thus be styled over and over again all day long.

Pomades usually make due without alcohol and synthetic styling polymers (=film-forming polymers) and therefore do not dry out the hair. Usually, pomade is an ointment-like substance composed of various fats and exists based on oil and water. The latter is more flexible and easier to remove. Oil-based products hold the hairstyle better, but are more difficult to remove from the hair.

There is still a need for styling products for head and/or beard hair that impart high holding performance to treated hairs, without including synthetic, film-forming polymers. In addition, these styling products should thus give the treated hair a natural, non-greasy or oily shine.

### BRIEF SUMMARY

This object is achieved by a cosmetic composition for the temporary deformation of hair, comprising, in each case based on the total weight of the composition, a) from about 20 to about 60% by weight of water, b) from about 1 to about 20% by weight of at least one oil, and c) from about 20 to about 65% by weight of at least one alkoxylated fatty alcohol.

Surprisingly, it has been found that such a cosmetic composition for temporary deformation of hair, despite the absence of synthetic, film-forming polymers, thus imparts a high holding performance to treated hair, without making the hair appear oily or greasy. In addition, such a composition can be distributed in the hair very well and evenly.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The cosmetic composition for temporary deformation of hair contains water as the first essential ingredient. The amount of water is at least about 20% by weight, based on the total weight of the composition. Accordingly, it is a water-based cosmetic composition. The amount of water in the cosmetic composition is preferably at least about 30% by weight and more preferably at least about 35% by weight, in each case based on the total weight of the cosmetic composition. Accordingly, particularly preferred cosmetic compositions amount of water is from about 30 to about 60% by weight of water and more preferably from about 35 to about 60% by weight, in each case based on the total weight of the cosmetic composition.

The cosmetic composition contains at least one oil as a second essential ingredient.

In the context of this application, an oil refers to a compound that is water-immiscible or only slightly miscible and liquid at about 25° C.

Suitable oils are selected from the esters of linear or branched saturated or unsaturated fatty alcohols having from 2-30 carbon atoms with linear or branched saturated or unsaturated fatty acids having from 2 to 30 carbon atoms which can be hydroxylated. These include cetyl 2-ethylhexanoate, 2-hexyldecyl stearate (for example Eutanol® G 16 S), 2-hexyldecyl laurate, isodecylneopentanoate, isononyl isononanoate, 2-ethylhexyl palmitate (for example, Cegesoft® C 24) and 2-ethylhexyl stearate (for example, Cetiol® 868). Also preferred are isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyl octanoic acid-2-butyl octanoate, diisotridecyl acetate, n-butyl stearate, n-hexyl laurate, n-decyl oleate, oleyl oleate, oleyl erucate, erucyl oleate, erucyl erucate, ethylene glycol dioleate and ethylene glycol dipalmitate.

Further preferred oils are selected from natural and synthetic hydrocarbons, more preferably from mineral oils, paraffin oils, C₁₈-C₃₀ isoparaffins, in particular isoeicosane, polyisobutenes and polydecenes, for example, which are available, for example, under the name Emery® 3004, 3006, 3010 or under the name Ethylflo® from Albemarle or Nexbase®2004G from Nestle, further selected from C₈-C₁₆ isoparaffins, in particular from isodecane, isododecane, isotetradecane and isohexadecane and mixtures thereof, as well as 1,3-di-(2-ethylhexyl)-cyclohexane (available, for example, under the trade name Cetiol® S from BASF).

Further preferred oils are selected from the benzoic acid esters of linear or branched C8-22 alkanols. Particularly preferred are benzoic acid C12-C15 alkyl esters, for example, available as a commercial product Finsolv® TN, benzoic acid isostearyl ester, for example, available as a commercial product Finsolv® SB, ethylhexyl benzoate, for example, available as a commercial product Finsolv® EB, and benzoic acid octyldodecyl ester, for example, available as a commercial product Finsolv® BOD.

Further preferred oils are selected from fatty alcohols having 6 to 30 carbon atoms which are unsaturated or branched and saturated or branched and unsaturated. The branched alcohols are also often referred to as Guerbet alcohols, since they are obtainable by the Guerbet reaction. Preferred alcohol oils are 2-hexyldecanol (Eutanol® G 16), 2-octyldodecanol (Eutanol® G), 2-ethylhexyl alcohol and isostearyl alcohol.

Further preferred oils are selected from mixtures of Guerbet alcohols and Guerbet alcohol esters, for example, the commercial product Cetiol® PGL (2-hexyldecanol and 2-hexyldecyl laurate).

Further preferred oils are selected from the triglycerides (=triple esters of glycerol) of linear or branched, saturated or unsaturated, optionally hydroxylated C8-30 fatty acids. Particularly referred can be the use of natural oils, for example, amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderflower seed oil, currant seed oil, jojoba oil, coconut oil, linseed oil, macadamia nut oil, corn oil, almond oil, marula oil, evening primrose oil, olive oil, palm oil, palm kernel oil, Brazil nut oil, pecan oil, peach kernel oil, rapeseed oil, castor oil, sea buckthom fruit oil, sea buckthom seed oil, sesame oil, soybean oil, sunflower oil, grapeseed oil, walnut oil, wild rose oil, wheat germ oil and the like. However, synthetic triglyceride oils are also preferred, in particular capric/caprylic triglycerides, for example, the commercial products Myritol® 318, Myritol® 331 (BASF) or Miglyol® 812 (Hils) with unbranched fatty acid radicals and glyceryl triisostearin with branched fatty acid radicals.

Further preferred oils are selected from the dicarboxylic acid esters of linear or branched C2-C10 alkanols, in particular diisopropyl adipate, di-n-butyl adipate, di-(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate.

Further preferred oils are selected from the addition products of from about 1 to about 5 propylene oxide units of mono- or multivalent C₈-C₂₂ alkanols such as octanol, decanol, decanediol, lauryl alcohol, myristyl alcohol and stearyl alcohol, for example, PPG-2 myristyl ether and PPG-3 myristyl ether (Witconol® APM).

Further preferred oils are selected from the addition products of at least 6 ethylene oxide and/or propylene oxide units to mono- or multivalent C₃-C₂₂ alkanols such as glycerol, butanol, butanediol, myristyl alcohol, and stearyl alcohol, which can be esterified if desired, for example, PPG-14 butyl ether (Ucon Fluid® AP), PPG-9 butyl ether (Breox® B25), PPG-10-butanediol (Macol® 57), PPG-15 stearyl ether (Arlamol® E) and glycereth-7-diisononanoate.

Further preferred oils are selected from the C₈-C₂₂ fatty alcohol esters of monovalent or multivalent C₂-C₇ hydroxycarboxylic acids, in particular the esters of glycolic acid, lactic acid, malic acid, tartaric acid, citric acid and salicylic acid. Such esters based on linear C14/15-alkanols, for example, C12-C15 alkyl lactate, and of in 2-positioned branched C12/13 alkanols are to be referred to under the trademark Cosmacol® from the company Nordmann, Rassmann GmbH & Co, Hamburg, in particular the commercial products Cosmacol® ESI, Cosmacol® EMI and Cosmacol® ETI.

Further preferred oils are selected from the symmetrical, asymmetrical or cyclic esters of carbonic acid with C₃₋₂₂ alkanols, C₃₋₂₂ alkanediols or C₃₋₂₂ alkanetriols, for example, dicaprylyl carbonate (Cetiol® CC) or glycerol carbonate.

Further oils which can be preferred are selected from the esters of dimers of unsaturated C₁₂-C₂₂ fatty acids (dimer fatty acids) with monovalent linear, branched or cyclic C₂-C₁₈ alkanols or with polyvalent linear or branched C₂-C₆ alkanols.

Further oils which are suitable are selected from silicone oils including, for example, dialkyl and alkylaryl siloxanes such as cyclopentasiloxane, cyclohexasiloxane, dimethylpolysiloxane and methylphenylpolysiloxane, but also hexamethyldisiloxane, octamethyltrisiloxane and decamethyltetrasiloxane. Preferred can be volatile silicone oils, which can be cyclic, such as octamethylcyclotetrasiloxane, decamethylcyclopentasiloxane and dodecamethylcyclohexasiloxane, and mixtures thereof, such as those contained in commercial products DC 244, 245, 344 and 345 from Dow Corning. Likewise suitable are volatile linear silicone oils, in particular hexamethyldisiloxane (L₂), octamethyltrisiloxane (L₃), decamethyltetrasiloxane (L₄) and any mixtures of two or three of L₂, L₃ and/or L₄, preferably mixtures as are available, for example, in the commercial products DC 2-1184, Dow Corning® 200 (about 0.65 cSt) and Dow Corning® 200 (about 1.5 cSt) from Dow Corning. Preferred nonvolatile silicone oils are selected from higher molecular weight linear dimethylpolysiloxanes, commercially available, for example, under the name Dow Corning®190, Dow Corning® 200 fluid having kinematic viscosities (about 25° C.) in the range of from about 5-100 cSt, preferably from about 5-50 cSt or also from about 5-10 cSt, and dimethylpolysiloxane having a kinematic viscosity (about 25° C.) of about 350 cSt. It can be extremely preferable to use mixtures of the aforementioned oils.

It is preferred that the oil comprises an oil of natural origin. It is highly preferred that the oil comprises a vegetable oil, in particular coconut oil. Coconut oil has a melting point in the range of from about 23 to about 26° C. and is one of the oils in the context of this application.

The amount of the at least one oil can be from about 0.25 to about 20% by weight, more preferably from about 0.5 to about 15% by weight, still more preferably from about 0.75 to about 10% by weight, and most preferably from about 1 to about 5% by weight, in each case based on the total amount of cosmetic composition.

The cosmetic composition contains at least one alkoxylated fatty alcohol as a third essential ingredient.

An “alkoxylated fatty alcohol” is any fatty alcohol having a carbon chain of C5 or greater and further comprising at least one alkoxy group. The fatty alcohol can be selected, for example, from C₉-C₁₁ fatty alcohols, C₁₂-C₁₃ fatty alcohols, C₁₂-C₁₅ fatty alcohols, C₁₂-C₁₆ fatty alcohols, C₁₄-C₁₅ fatty alcohols, arachidyl alcohol, behenyl alcohol, caprylic alcohol, cetearyl alcohol, cetyl alcohol, coconut alcohol, decyl alcohol, (hydrogenated) tallow alcohol, lauryl alcohol, myristyl alcohol, oleyl alcohol, palm alcohol, palm kernel alcohol, stearyl alcohol, and tridecyl alcohol.

The alkoxy group can in particular comprise ethoxy and/or propoxy and/or butoxy groups. Most preferably, the alkoxylated fatty alcohol is an ethoxylated fatty alcohol.

The at least one alkoxy group of the “alkoxylated fatty alcohol” can be derived, for example, from an alkoxylation reaction with alkylene oxide, in particular ethylene oxide and/or propylene oxide.

It is preferred that the at least one alkoxylated fatty alcohol is selected from the group including Ceteareth-2, Ceteareth-3, Ceteareth-4, Ceteareth-5, Ceteareth-6, Ceteareth-7, Ceteareth-8, Ceteareth-9, Ceteareth-10, Ceteareth-11, Ceteareth-12, Ceteareth-13, Ceteareth-14, Ceteareth-15, Ceteareth-16, Ceteareth-17, Ceteareth-18, Ceteareth-20, Ceteareth-22, Ceteareth-23, Ceteareth-24, Ceteareth-25, Ceteareth-27, Ceteareth-28, Ceteareth-29, Ceteareth-30, Ceteareth-33, Ceteareth-34, Ceteareth-40, Ceteareth-50, Ceteareth-55, Ceteareth-60, Ceteareth-80, Ceteareth-100, Laureth-1, Laureth-2, Laureth-3, Laureth-4, Laureth-5, Laureth-6, Laureth-7, Laureth-8, Laureth-9, Laureth-10, Laureth-11, Laureth-12, Laureth-13, Laureth-14, Laureth-15, Laureth-16, Laureth-20, Laureth-23, Laureth-25, Laureth-30, Laureth-40, Deceth-3, Deceth-5, Oleth-5, Oleth-30, Steareth-2, Steareth-4, Steareth-6, Steareth-7, Steareth-10, Steareth-11, Steareth-13, Steareth-14, Steareth-15, Steareth-20, Steareth-21, Steareth-25, Steareth-27, Steareth-30, Steareth-40, Steareth-50, Steareth-100 and mixtures thereof.

The term Ceteareth-2, for example, stands for a C₁₆-C₁₈ fatty alcohol having an average of 2 ethylene oxide units per molecule.

In a very particularly preferred embodiment of the cosmetic composition, the at least one alkoxylated fatty alcohol comprises Ceteareth-25. In a very particularly preferred embodiment of the cosmetic composition, the at least one alkoxylated fatty alcohol is Ceteareth-25.

The amount of the at least one alkoxylated fatty alcohol is preferably from about 20 to about 50% by weight and more preferably from about 25 to about 40% by weight, in each case based on the total amount of cosmetic composition.

In an extremely preferred embodiment of the cosmetic composition, it contains from about 20 to about 50% by weight and more preferably from about 25 to about 40% by weight, in each case based on the total amount of cosmetic composition, of Ceteareth-25 as at least one alkoxylated fatty alcohol.

The cosmetic composition is preferably free of synthetic, film-forming polymers. “Free from” means that the cosmetic composition contains at most about 0.2% by weight and preferably 0% by weight, in each case based on the total weight of the composition, of synthetic, film-forming polymers.

In a preferred embodiment, the composition further comprises at least one multivalent alcohol.

The multivalent alcohol is preferably selected from the group including 1,2-propanediol, 1,3-propanediol, 2-methyl-1,3-propanediol, glycerol, 1,2-butylene glycol, 1,3-butylene glycol, 1,4-butylene glycol, 1,2-pentanediol, 1,5-pentanediol, 1,2-hexanediol, 1,6-hexanediol, 2-methyl-2,4-pentanediol, 1,2,6-hexanetriol, 1,2-octanediol, 1,8-octanediol, dipropylene glycol, tripropylene glycol, diglycerol, triglycerol, erythritol, sorbitol, cis-1,4-dimethylolcyclohexane, trans-1,4-dimethylolcyclohexane, any isomeric mixtures of cis- and trans-1,4-dimethylolcyclohexane, PEG-3, PEG-4, PEG-6, PEG-7, PEG-8, PEG-9, PEG-10, PEG-12, PEG-14, PEG-16, PEG-18, and PEG-20, and mixtures hereof.

Particularly preferred multivalent alcohols are selected from 1,2-propanediol, glycerol, 1,3-butylene glycol, 1,6-hexanediol, and sorbitol, and mixtures thereof. Most preferred is glycerol or a mixture of glycerol and 1,2-propanediol.

The amount of the at least one multivalent alcohol is preferably from about 4 to about 18% by weight and more preferably from about 6 to about 15% by weight, in each case based on the total amount of cosmetic composition.

In a further preferred embodiment, the composition further comprises at least one emulsifier.

The at least one emulsifier is preferably nonionic and an optionally polyethoxylated fatty acid polyol ester of the type of fatty acid glycol monoester, fatty acid glyceryl monoester and sorbitan fatty acid ester. Preferred examples of optionally polyethoxylated fatty acid glycol monoesters are PEG-100 stearates, PEG-90 stearates, PEG-50 stearates, PEG-30 stearates, PEG-10 stearates, PEG-7 stearates, PEG-100 oleates, PEG-90 oleates, PEG-50 stearates, PEG-30 oleates, PEG-10 oleates, PEG-7 oleates, PEG-100 cocoates, PEG-90 cocoates, PEG-50 cocoates, PEG-30 cocoates, PEG-10 cocoates, PEG-7 cocoates.

Preferred examples of optionally polyethoxylated fatty acid glyceryl monoesters are glyceryl stearates, PEG-30 glyceryl stearates, PEG-20 glyceryl stearates, glyceryl oleates, glyceryl cocoates, glyceryl palmitates, PEG-18 glyceryl oleates, PEG-18 glyceryl cocoates, PEG-7 glyceryl cocoates, PEG-30 glyceryl cocoates, PEG-80 glyceryl cocoates.

Preferred examples of optionally polyethoxylated sorbitan fatty acid esters are those known under the INCI names Polysorbate-20 (sorbitan monolaurate+20 EO), Polysorbate-60 (sorbitan monostearate+20 EO), Polysorbate-65 (sorbitan tristearate+20 EO), Polysorbate-80 (sorbitan monooleate+20 EO), Polysorbate-85 (sorbitan trioleate+20 EO).

Polyethoxylated fatty acid glyceryl monoesters are preferred emulsifiers of the fatty acid polyol esters mentioned. A most preferred emulsifier is PEG-7 Glyceryl Cocoate.

The amount of the at least one emulsifier can be from about 1 to about 10% by weight and more preferably from about 2 to about 6% by weight, in each case based on the total amount of cosmetic composition.

The cosmetic composition exists in particular in the form of a pomade and is suitable for temporary deformation of hair, in particular head and/or beard hair. The cosmetic composition exists in particular in the form of a water-based pomade, which can be easily removed from the hair treated therewith, for example, by using water and/or a hair cleaning composition such as a shampoo.

The following example is intended to illustrate the subject matter of the present disclosure without limiting it in any way.

### Example

| | % by weight (active |
|---|---|
| Ingredient | substance) |
| Ceteareth-25 | 30 |
| Coconut oil | 2 |
| 1,2-propanediol | 1 |
| Glycerol | 10 |
| PEG-7 Glyceryl Cocoate | 5 |
| Citric acid | 0.1 |
| Preservative | 1 |
| Perfume | 1 |
| Water | to 100 |

The formulation as contemplated herein was applied to subjects for fixing the shape of the hairstyle by hand. The hair received a natural shine and a strong, but flexible hold of the hairstyle. The hairs of the subjects in this case were always soft, supple and combable. In addition, the subjects had a positive hair feeling.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition for temporary deformation of hair, comprising, in each case based on the total weight of the composition,
a) from about 20 to about 60% by weight of water,
b) from about 0.5 to about 20% by weight of at least one oil, and
c) from about 20 to about 65% by weight of at least one alkoxylated fatty alcohol.

2. The cosmetic composition according to claim 1, wherein the at least one alkoxylated fatty alcohol is selected from the group consisting of Ceteareth-2, Ceteareth-3, Ceteareth-4, Ceteareth-5, Ceteareth-6, Ceteareth-7, Ceteareth-8, Ceteareth-9, Ceteareth-10, Ceteareth-11, Ceteareth-12, Ceteareth-13, Ceteareth-14, Ceteareth-15, Ceteareth-16, Ceteareth-17, Ceteareth-18, Ceteareth-20, Ceteareth-22, Ceteareth-23, Ceteareth-24, Ceteareth-25, Ceteareth-27, Ceteareth-28, Ceteareth-29, Ceteareth-30, Ceteareth-33, Ceteareth-34, Ceteareth-40, Ceteareth-50, Ceteareth-55, Ceteareth-60, Ceteareth-80, Ceteareth-100, Laureth-1, Laureth-2, Laureth-3, Laureth-4, Laureth-5, Laureth-6, Laureth-7, Laureth-8, Laureth-9, Laureth-10, Laureth-11, Laureth-12, Laureth-13, Laureth-14, Laureth-15, Laureth-16, Laureth-20, Laureth-23, Laureth-25, Laureth-30, Laureth-40, Deceth-3, Deceth-5, Oleth-5, Oleth-30, Steareth-2, Steareth-4, Steareth-6, Steareth-7, Steareth-10, Steareth-11, Steareth-13, Steareth-14, Steareth-15, Steareth-20, Steareth-21, Steareth-25, Steareth-27, Steareth-30, Steareth-40, Steareth-50, Steareth-100 and mixtures thereof.

3. The cosmetic composition according to claim 1, wherein the at least one alkoxylated fatty alcohol comprises Ceteareth-25.

4. The cosmetic composition according to claim 1, wherein the at least one alkoxylated fatty alcohol is Ceteareth-25.

5. The cosmetic composition according to claim 1, wherein the at least one oil is selected from the group consisting of
esters of linear or branched saturated or unsaturated fatty alcohols having 2 to 30 carbon atoms with linear or branched saturated or unsaturated fatty acids having 2 to 30 carbon atoms,
natural and synthetic hydrocarbons,
benzoic acid esters of linear or branched C₈₋₂₂ alkanols,
fatty alcohols having 6 to 30 carbon atoms, which are unsaturated or branched and saturated or branched and unsaturated
triglycerides,
dicarboxylic acid esters of linear or branched C₂-C₁₀ alkanols,
addition products of from about 1 to about 5 propylene oxide units of mono- or multivalent C₅-C₂₂ alkanols,
addition products of at least about 6 ethylene oxide and/or propylene oxide units to mono- or multivalent C₃-C₂₂ alkanols,
C₅-C₂₂ fatty alcohol esters of monovalent or multivalent C₂-C₇ hydroxycarboxylic acids,
symmetric, unsymmetrical or cyclic esters of carbonic acid with C₃₋₂₂ alkanols, C₃₋₂₂ alkanediols or C₃₋₂₂ alkanetriols,
esters of dimers of unsaturated C₁₂-C₂₂ fatty acids (dimer fatty acids) with monovalent linear, branched or cyclic C₂-C₁₈ alkanols or with polyvalent linear or branched C₂-C₆ alkanols,
silicone oils, and
mixtures thereof.

6. The cosmetic composition according to claim 5, wherein the at least one oil is selected from the group of triglycerides.

7. The cosmetic composition according to claim 1, wherein the composition further comprises at least one emulsifier.

8. The cosmetic composition according to claim 1, wherein the composition further comprises at least one multivalent alcohol.

9. A use of a cosmetic composition according to claim 1 for temporary deformation of keratinic fibers.

10. A method for temporary deformation of hair, the method comprising applying a cosmetic composition according to claim 1 to the hair, wherein the hair is temporarily fixed in shape.
